(19) United States
(12) Patent Application Publication
(10) Pub. No.: US 2026/0091127 A1
(43) Pub. Date: Apr. 2, 2026

(54) JANUS PARTICLE-EMBEDDED MATERIALS FOR ANTIMICROBIAL APPLICATIONS

(71) Applicants: The Trustees of Indiana University, Bloomington, IN (US); The University of Osaka, Osaka (JP)

(72) Inventors: Martijn Zwama, Bloomington, IN (US); Yan Yu, Bloomington, IN (US); Swagata Bhattacharyya, Bloomington, IN (US); Kunihiko Nishino, Bloomington, IN (US)

(21) Appl. No.: 19/344,327

(22) Filed: Sep. 29, 2025

Related U.S. Application Data
(60) Provisional application No. 63/700,985, filed on Sep. 30, 2024.

(51) Int. Cl. A61K 47/69; A61K 38/12; A61P 31/04

(52) U.S. Cl. CPC A61K 47/6929; A61K 38/12; A61P 31/04

(57) ABSTRACT

Antimicrobial Janus nanoparticles (NPs) having distinct hydrophobic and polycationic surfaces are embedded in crosslinked polymers. These encapsulated Janus NPs have increased stabilization, to allow for long-term effectiveness. The encapsulated Janus NPs enhance traditional antibiotics and reduce the necessary dosage of those traditional antibiotics to inhibit growth of muti-drug resistant bacteria.

### STATEMENT OF GOVERNMENTAL RIGHTS

This invention was made with government support under 2153891 awarded by the National Science Foundation. The government has certain rights in the invention.

### FIELD OF THE INVENTION

The present disclosure relates to nanoparticles, particularly in the field of antimicrobials and antimicrobial treatments.

### BACKGROUND

The emergence of multidrug-resistant pathogenic infections has escalated into a critical global healthcare issue. With bacteria evolving resistance to multiple antibiotics, our once robust arsenal of antibiotics to combat infectious diseases is rapidly depleting. There is a need to develop innovative classes of antimicrobial materials that can effectively counteract the effect of drug resistance. There is also a need to develop ways to utilize currently available antibiotics more effectively again pathogens.

Previously, we described a novel antibacterial nanoparticle (NPs) that broke with state-of-the-art teachings of uniform surface chemistry on the nanoparticle. Our novel Janus NPs (JNPs, JPs) feature hydrophobic ligands and polycationic ligands on opposite hemispheres, which make them orders of magnitudes more effective against a broad range of bacteria. However, a key obstacle to the broad clinical application of these antimicrobial Janus NPs has been its instability in biological fluids for a prolonged period. Janus NPs have shown susceptibility to aggregation in biological fluids. This instability manifests at high concentrations and/or elevated temperatures, particularly over extended time periods exceeding several days.

While progress has been made in the development of improved antibacterial nanoparticles through the use of Janus NPs, further improvements are still needed to address stability issues of antimicrobial Janus NPs to facilitate their use in practical antibacterial applications. Aspects of the invention disclosed herein address this need.

### SUMMARY OF THE INVENTION

A first aspect of the invention includes an antimicrobial material, comprising: a Janus nanoparticle, a first antibiotic and a cross-linked polymer, wherein the Janus nanoparticle comprises a nanoparticle having a plurality of hydrophobic moieties attached to a first hemisphere of the nanoparticle and a plurality of charged moieties attached to the opposite hemisphere of the nanoparticle, and wherein the Janus nanoparticle is embedded within the cross-linked polymer.

A second aspect of the invention includes a method of administering an antimicrobial to a subject in need thereof, the method comprising administering to said subject an antimicrobial material comprising a Janus nanoparticle, a first antibiotic and a cross-linked polymer, wherein the Janus nanoparticle comprises a nanoparticle having a plurality of hydrophobic moieties attached to a first hemisphere of the nanoparticle and a plurality of charged moieties attached to the opposite hemisphere of the nanoparticle, and wherein the Janus nanoparticle is embedded within the cross-linked polymer.

A third aspect of the invention includes the antimicrobial material where the charged moiety of the embedded Janus nanoparticle is a second antibiotic.

A fourth aspect of the invention wherein the first antibiotic and the second antibiotic are different.

A fifth aspect of the invention includes a wound dressing incorporating the antimicrobial material.

A sixth aspect of the invention includes administering the antimicrobial material by applying the antimicrobial material topically to the subject.

A seventh aspect of the invention includes administering the antimicrobial material by applying the antimicrobial material topically to a surgical wound.

An eighth aspect of the invention includes administering the antimicrobial material by applying the antimicrobial material topically to a bacterial infection present on the skin of a subject.

A ninth aspect of the invention includes administering the antimicrobial material within a wound.

A tenth aspect of the invention includes administering an antimicrobial material where the therapeutically effective dose of Janus nanoparticles contained in the antimicrobial material is within the picomolar to micron-molar range.

An eleventh aspect of the invention includes administering an antimicrobial material where the bacterial infection is caused by at least one bacterium selected from the group consisting of: Gram-negative and Gram-positive bacteria.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A Illustration of an exemplary antimicrobial Janus nanoparticle (Janus NP or JP) and its fabrication procedure as described in Example 1. In this example, the charged moiety is colistin (col/pho JP).

FIG. 1B (Left) illustration showing interaction of JPs embedded inside a crosslinked polymer gel with bacteria. (Right) illustration showing disruption of bacterial membranes by JPs.

FIG. 2A Images of hydrophobic/colistin Janus NPs (JP-col) at high (photo on left) and low (photo on right) concentrations in HEPES buffer (2 mM HEPES pH 7.2, 25 mM NaCl) after incubation for 2 hours at 37° C. while shaking.

FIG. 2B Image of Janus NPs adhered to walls of plastic tubes and microplate wells after several hours of incubation and shaking.

FIG. 3A Image of JP-col dispersed and stabilized in warm LB agar at 70° C. after sonication.

FIG. 3B Images of three concentrations of JP-col mixed with LB agar. Janus NP concentration is: 32 pM (top row), 8 pM (middle row), 0 pM (bottom row).

FIG. 4A Image of A. baumannii growth on agar encapsulating different concentrations of Janus NPs in LB supplemented with different concentrations of CLX (32 to 1024 μg/mL). Concentrations of Janus NPs encapsulated in agar: 32 pM (top), 8 pM (middle), and 0 pM (bottom).

FIG. 4B Images showing E. coli growth on agar encapsulating 32 pM Janus NPs (top row) compared to the cells grown on regular agar. All samples were in LB supplemented with different concentrations of ethidium bromide (EB) (32 to 1024 μg/mL).

FIG. 5A Image of physical wells (right) from checkerboard experiment testing effect of JNP (pM) on antimicrobial activity of erythromycin (μg/mL) against A. baumannii ATCC19606 and schematic representation (left) of those results, with light grey indicating complete growth inhibition; medium grey indicating partial inhibition; and dark grey indicating full bacterial growth.

FIG. 5B Image of physical wells (right) from checkerboard experiment testing effect of UNP (pM) on antimicrobial activity of erythromycin (μg/mL) against A. baumannii ATCC19606 and schematic representation (left) of those results, with light grey indicating complete growth inhibition; medium grey indicating partial inhibition; and dark grey indicating full bacterial growth.

FIG. 6 Image of physical wells (right) from checkerboard experiment testing effect of JNP (pM) on antimicrobial activity of novobiocin (μg/mL) against A. baumannii ATCC19606 and schematic representation (left) of those results, with light grey indicating complete growth inhibition; medium grey indicating partial inhibition; and dark grey indicating full bacterial growth.

FIG. 7A Image of physical wells (right) from checkerboard experiment testing effect of JNP (pM) on antimicrobial activity of erythromycin (μg/mL) against A. baumannii isolate A42-2 and schematic representation (left) of those results, with light grey indicating complete growth inhibition; medium grey indicating partial inhibition; and dark grey indicating full bacterial growth.

FIG. 7B Image of physical wells (right) from checkerboard experiment testing effect of JNP (pM) on antimicrobial activity of novobiocin (μg/mL) against A. baumannii isolate A42-2 and schematic representation (left) of those results, with light grey indicating complete growth inhibition; medium grey indicating partial inhibition; and dark grey indicating full bacterial growth.

FIG. 8A Schematic representation of checkerboard experiment testing effect of JNP (pM) on antimicrobial activity of erythromycin (μg/mL) (left) or ethidium bromide (μg/mL) (right) against E. coli MG1655, with light grey indicating complete growth inhibition; medium grey indicating partial inhibition; and dark grey indicating full bacterial growth.

FIG. 8B Schematic representation of checkerboard experiment testing effect of UNP (pM) on antimicrobial activity of erythromycin (μg/mL) (left) or ethidium bromide (μg/mL) (right) against E. coli MG1655, with light grey indicating complete growth inhibition; medium grey indicating partial inhibition; and dark grey indicating full bacterial growth.

FIG. 9 Scanning electron microscopy (SEM) images of Janus nanoparticles (JNPs) (left) and JNP-embedded agar gel (right).

FIG. 10 Image of physical wells (bottom) from checkerboard experiment testing effect of JNP (pM) on antimicrobial activity of cloxacillin (μg/mL) against A. baumannii in solid agar and schematic representation (top) of those results, with light grey indicating complete growth inhibition; medium grey indicating partial inhibition; and dark grey indicating full bacterial growth.

### DETAILED DESCRIPTION

We describe here a new type of antimicrobial material in which antimicrobial Janus nanoparticles (NPs) are embedded in crosslinked polymers. Also shown herein are methods for creating these antimicrobial materials as well as methods for preserving the antimicrobial properties and long-term stability of Janus NPs across temperature variations through encapsulation of the Janus NPs within a polymer gel. This innovative antimicrobial material not only demonstrated antibacterial activity but also demonstrated remarkable effectiveness in reducing bacterial resistance to traditional antibiotics. When employed in conjunction with traditional antibiotics, the antibacterial materials disclosed herein significantly lower the required antibiotic dosage required for the traditional antibiotic to effectively inhibit the growth of bacteria. This improved (and often synergistic) increased inhibitory effect of the embedded JPs was particularly remarkable in that this effect parallels that of JNPs in suspension, where the nanoparticles can interact with bacterial cells from all directions. The present invention offers a promising strategy for combating antibiotic resistance not only through the novel antibiotic activity of the Janus NP, but also by potentiating the effects of existing antibiotics with a lower dosage requirement.

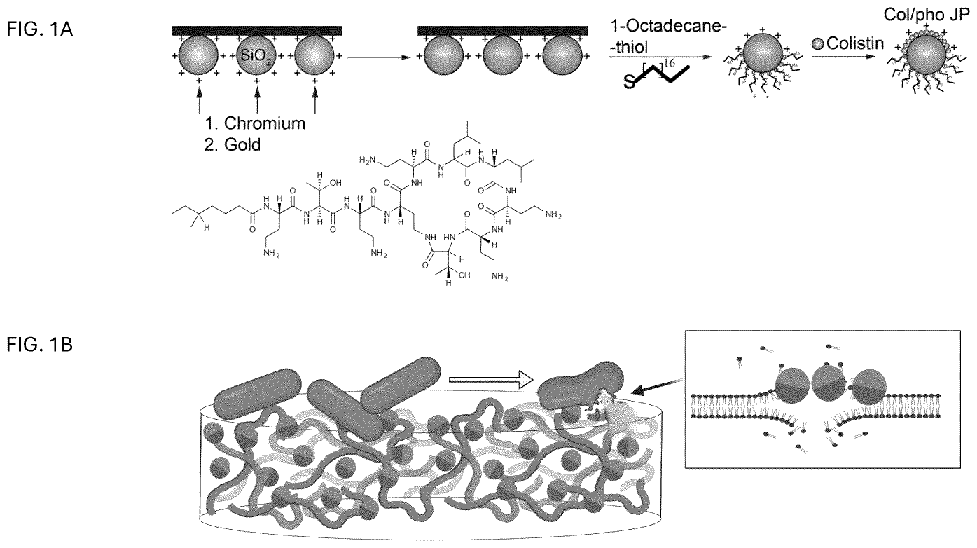

Janus nanoparticles, named after the Roman god Janus with two faces in opposite directions, are anisotropic nanoparticles with two distinct hemispheres. Specifically, the surface of the Janus nanoparticles (also referred to herein as Janus NPs or JPs) is divided into two hemispheres. One hemisphere of the Janus NP has hydrophobic moieties (and/or ligands) attached and the opposite hemisphere of the Janus NP has charge moieties (and/or ligands) attached. (FIG. 1A). Thus, the hydrophobic and charged ligands are spatially separated onto two separate hemispheres on the Janus nanoparticles. This allows the nanoparticles to be attracted to lipid membranes through electrostatic interaction without interference from the hydrophobic ligands, and then reorient to insert into bio-membranes from their hydrophobic hemisphere without interference from the charged hemisphere. (FIG. 1B).

In some embodiments of the Janus particles, the hydrophobic hemisphere includes hydrophobic ligands including, but are not limited to, hydrophobic alkyl chains. Those of skill in the art will appreciate that the hydrophobic alkyl chains may include linear chains, branched chains, or polymers. Preferred hydrophobic alkyl moieties for use in the Janus nanoparticles include 4 or more carbon-carbon bonds with alkyl moieties having a backbone of greater than 4 carbon-carbon bonds in length most preferred.

In some embodiments of the Janus particles, the charged moiety may include, but is not limited to, an antibiotic such as cationic lipopeptides (such as polymyxin B and polymyxin E (also known as colistin), antimicrobial peptides (such as ambicin, also known as nisin), Gramicidins (such as Gramicidin S), Ceragenins, Peptidomimetic-based antimicrobial cationic amphiphiles, Benzophenone-based antimicrobial cationic amphiphiles, Xanthone-based antimicrobial cationic amphiphiles, Aminoglycoside-derived antimicrobial cationic amphiphiles, or steroid antibiotics. One of skill in the art will appreciate that a variety of antibiotic molecules may be used in the disclosed Janus particles where the antibiotic exhibits a positive charge. The disclosed Janus nanoparticles may also include a cationic polymer as the charged moiety to be placed opposite the hydrophobic moiety. Cationic polymers may include cationic poly(amidoamine), cationic linear polymers, cationic branched polymers, cationic dendrimers, cationic polypeptide, or other cationic nanoparticles.

Core particles for use in creating the antibacterial Janus nanoparticles of the invention include, but are not limited to, inorganic particles, polymeric particles, and metal particles. One of ordinary skill in the art would appreciate that a variety of materials capable of use as microparticles and/or nanoparticles may also be utilized as carrier particles in the disclosed invention. In one embodiment of the invention, the carrier particle is silica or polystyrene.

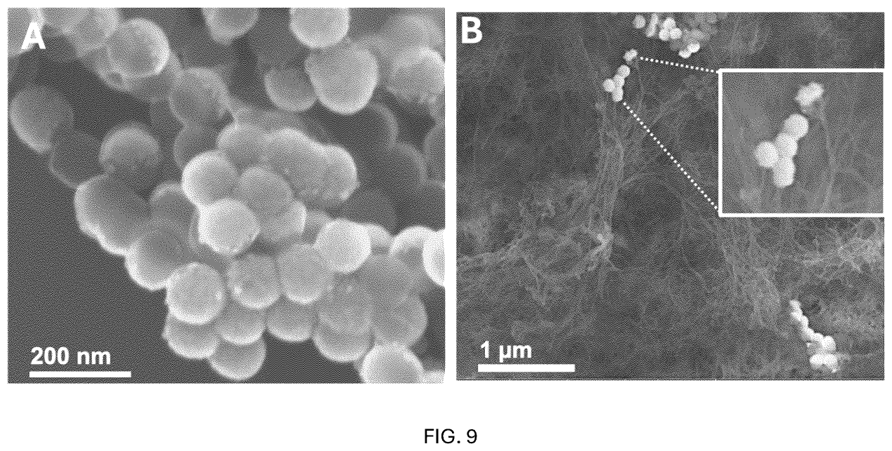

Because nanoparticles in solution are prone to aggregation and uneven distribution, described herein is an alternative delivery method to ensure stable and uniform exposure of bacterial cells to JNP. Altering the mode of nanoparticle administration: instead of liquid culture, JNPs were embedded directly into solid agar media supplemented with nutrients and antibiotics. As shown in FIG. 9, scanning electron microscope (SEM) images confirm the morphology of Janus NPs and their incorporation within the agar matrix.

Polymer Gels of the present invention include any kind of crosslinked polymer gel. Polymer gels of the present invention can be naturally derived, artificial or semi-synthetic. Polymers include alginates or chitosans as well as other non-toxic, biocompatible forming polymers such as collagen, or a derivative thereof. Polymers can also include cellulose polymers or derivatives of cellulose. Polymer gels can also include other FDA-approved biodegradable synthetic polymers such as polycaprolactone (PCL), polylactic acid (PLA), poly lactic-co-glycolic acid (PLGA), or combinations thereof. Polymer gels of the present invention also include biocompatible hydrogels. Preferably, the polymer gels are those with known biocompatibility to mammalian subjects such that they produce no toxic effect upon the subject. Janus NPs have shown to be effective in killing both Gram-negative and Gram-positive bacteria. As shown herein, Janus NPs embedded within a polymer gel were also effective in killing both Gram-negative and Gram-positive bacteria. Examples of Gram-negative bacteria susceptible to the disclosed Janus particles include, but are not limited to, strains of Escherichia coli and Vibrio cholerae. Examples of Gram-positive bacteria susceptible to the disclosed Janus particles include, but are not limited to, strains of Bacillus subtilis and Staphylococcus aureus.

The concentration of antimicrobial Janus particles needed to reduce the population of bacteria present (the EC50) is significantly less than that observed by prior art nanoparticles with uniform surface chemistry. This potency was observed in both Gram-negative and Gram-positive bacteria. Surprisingly, the potency of the antimicrobial JPs was not reduced by encapsulating the JPs in a hydrogel. As shown herein, antimicrobial materials containing picomolar and nanomolar quantities of antimicrobial Janus particles embedded within the polymer showed significant reduction in bacterial growth.

Furthermore, as shown herein, when used in conjunction with traditional antibiotics, the presence of antibacterial JP within the polymer gel significantly improves antibacterial potency and reduces the concentration of antibiotic needed to achieve equivalent or greater inhibition of bacterial growth.

To prepare the antimicrobial material of the present invention, the polymer is prepared and sterilized according to standard protocols. A stock solution of antibacterial JP is prepared. The final concentration of antibiotic JP for the stock solution is determined based upon the final concentration of JP desired in the polymer. The concentration of antibiotic in the final antimicrobial material should be below a concentration that could interfere with the polymerization and/or integrity of the polymer.

Optionally, the antibiotic JP stock solution may be sonicated for better dispersion of the nanoparticles within the solution. This may be performed using an ultrasonic bath or a low-power sonicator. However, excessive heat during sonication should be avoided.

The JP stock solution is then added to the polymer. Preferably, the JP and polymer are mixed or sonicated to ensure even dispersion of the JP particles throughout the polymer. The polymer/JP mixture may then be dispensed into a template, form, frame, die or container to form the final desired shape and thickness of the antibiotic material. Alternatively, the polymer/JP mixture may be sprayed onto a surface to create a sheet of antibiotic material or to create a coating on the surface.

Traditional antibiotics may also be incorporated into the polymer/JP mixture. The antibiotics may be added to the polymer/JP mixture prior to dispersion, or the antibiotics may be added to the antibiotic material after dispersion. The timing of the addition of the antibiotics to the polymer/JP mixture will depend upon the final form and use of the antibiotic material desired. Both fast-acting and slow-acting antibiotics may be incorporated into the polymer/JP mixture. Exemplary antibiotics include, but are not limited to ciprofloxacin (CIP), amikacin (AMK), meropenem (MER), moxifloxacin (MOX), cloxacillin (CLX), erythromycin (EM), rifampicin (RIF), tetracycline (TET), ethidium bromide (EB), novobiocin (NOV) and polymyxin B (PB). The concentration of antibiotic added to the polymer/JP mixture is 0.5 to 0.01 the minimum inhibitory concentration (MIC) (mg/mL) of the antibiotic needed to reach the same MIC alone.

The antimicrobial properties of the disclosed antibacterial materials may be utilized in killing bacteria on exposed surfaces of animal tissues as well as treating bacterial infections in and/or on a human or animal. Antimicrobial JP encapsulated polymer gels are suitable for biomedical applications, including wound dressings. Wound dressings incorporating the antimicrobial material described herein can be effective for use as general surgical wound dressing, burn dressing, donor site dressing, bedsore dressing, ulcer dressing and the like, as well as in dermatological applications.

Additionally, the antimicrobial JP encapsulated polymer gels may also be utilized in antibacterial coatings on exposed surfaces of inanimate objects. JP/polymer gel antibacterial coatings may be applied to a variety of materials and products, including medical devices, public surfaces, healthcare textiles, sportwear textiles, or filtration systems for water treatment.

The antimicrobial JP polymer gels described herein are a significant advance over prior nanoparticle-embedded gels. Compared to prior nanoparticle-embedded gels, the JP polymer gels require significantly less amount of particles to be embedded to achieve high levels of potency in inhibiting bacterial growth. The JP polymer gels inhibit the growth of multi-drug resistant bacteria, preventing biofilm growth.

### Abbreviations and Definitions

Unless states otherwise of clearly implied otherwise the term ‘therapeutic amount’ is the amount is an amount of the compound that either in a single dose or as part of course of treatment has a therapeutic effect on a patient.

Unless states otherwise of clearly implied otherwise the term ‘patient’ refers to either a human or an animal

### EXAMPLES

### Example 1: Nanoparticle Fabrication

Amphiphilic cationic Janus nanoparticles were fabricated using the techniques as previously described, in WO2023/154740, incorporated herein by reference. Cationic silica nanoparticles (100 nm in diameter) were drop cast onto piranha etched microscope slides to make a sub-monolayer of particles. An Edwards thermal evaporation system (Nanoscale Characterization Facility at Indiana University) was used to sequentially deposit thin layers of chromium (5 nm) and gold (25 nm) onto one hemisphere of the nanoparticles. Particle monolayers were immediately immersed in 2 mM 1-octadecanethiol for at least 12 h before use to make the gold caps on particles hydrophobic. Particles were sonicated off the microscope slides and subjected to differential centrifugation (4 times at 100×g, 4 times at 500×g) to remove metal bridging aggregates formed during thermal evaporation. Janus particle gold coating was assessed by scanning electron microscopy (Nanoscale Characterization Facility at Indiana University). Amphiphilic silica nanoparticles were made prior to colistin conjugation. Briefly, octadecyltrimethoxysilane and IM HCl were added dropwise to tetrahydrofuran (THF) to prepare a solution containing 22 mM octadecyltrimethoxysilane and 0.6 vol % of HCl. Cationic silica nanoparticles were resuspended in 8:1 (v/v) hexanes: octadecyltrimethoxysilane solution with vigorous stirring for 1 h at room temperature. The resulted amphiphilic silica nanoparticles were washed 3 times with ethanol and 3 times with water before further surface modification.

The cationic hemisphere of the Janus nanoparticles was conjugated with colistin (JP-col). Colistin is a cationic antibiotic molecule that is often use as a last resort to kill Gram-negative bacteria. (Available from Sigma Aldrich). Colistin is thought to work by binding lipopolysaccharide in the cell envelope.

To conjugate colistin, nanoparticles (amphiphilic Janus nanoparticles, amphiphilic cationic silica particles, or cationic silica nanoparticles) were washed 3 times with 10 mM HEPES buffer (pH 7.4) and resuspended with 10% glutaraldehyde in 10 mM HEPES buffer for 90 min at room temperature with gentle rotation. After glutaraldehyde activation of amine groups on the particle surface, the particles were washed three times with 10 mM HEPES buffer to remove excess glutaraldehyde. Particles were then resuspended with colistin (1 mg/mL in 10 mM HEPES buffer) for 60 min at room temperature with gentle rotation. Particles were washed three times again with 10 mM HEPES followed by three times washing with 2 mM HEPES 25 mM NaCl buffer to remove unreacted colistin. Particles were stored at 4° C. until use. Hydrodynamic radius and zeta potential of all particles were characterized using a Malvern Zetasizer (Nanoscale Characterization Facility at Indiana University). Concentration of particles was measured using Particle Metrix ZetaView (Nanoscale Characterization Facility at Indiana University).

JP-col were fabricated by coating one hemisphere of 100 nm aminated silica nanoparticles with chromium and then gold, which was subsequently conjugated with octadecanethiol to make the gold hemisphere more hydrophobic. Colistin was then conjugated to the nanoparticles using glutaraldehyde crosslinking of the primary amine groups on the silica hemisphere to L-diaminobutyric acid residues in colistin.

### Example 2: Agar/Janus Nanoparticle Preparation Protocol for Bacterial Susceptibility Testing

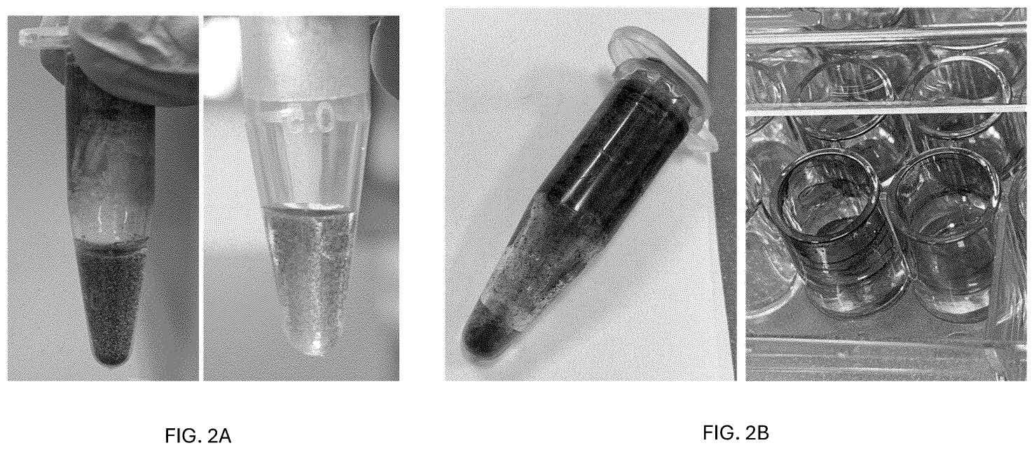

Agar was employed as a gelling agent to create a solid medium for bacteria growth. Traditional liquid growth media present limitations for Janus NP (JP) stability, as shown in FIG. 2A. JPs can also adhere to the sides of tubes and well plates during extended incubation at 37° C. under specific conditions (FIG. 2B). In this study, we utilized LB agar as an exemplary medium to optimize the bioaccessibility of JPs to bacteria. Other growth media can be employed if needed.

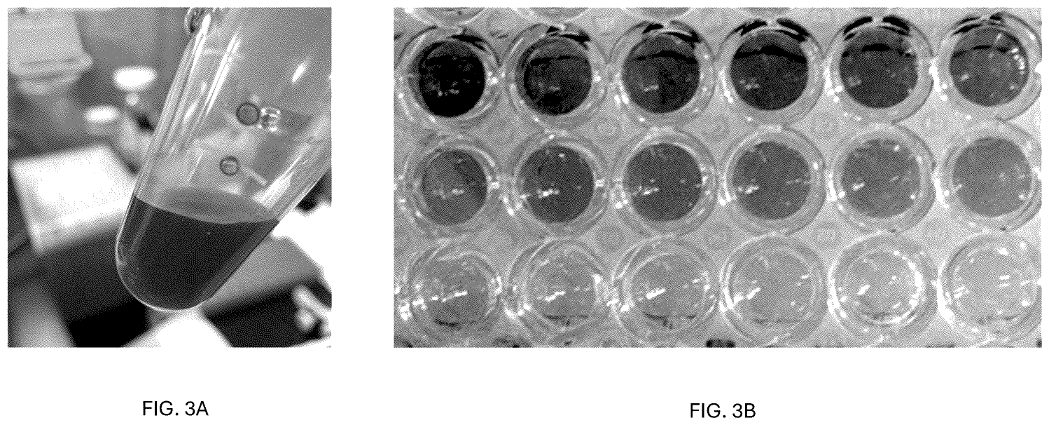

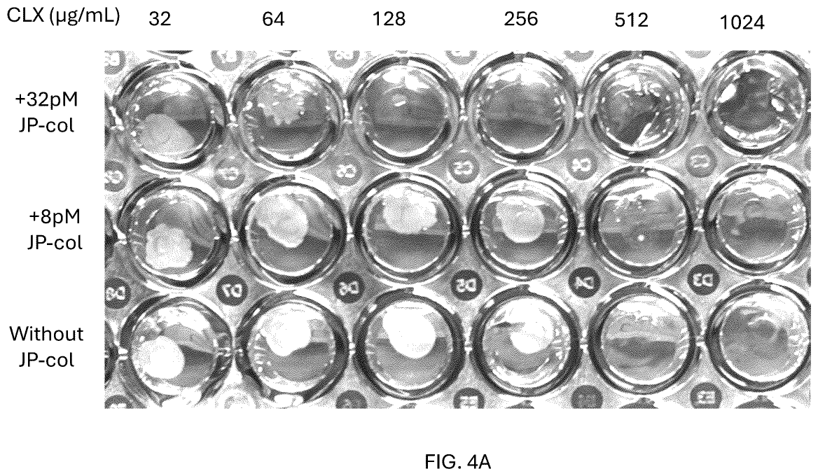

To optimize resource usage and minimize JP consumption, we conduct the experiments in a 96-well plate format, allowing for the preparation of smaller volumes (e.g., 200 μL) of agar/JP mixtures within each well. JPs containing colistin moieties (JP-col) as described above were incorporated into the agar (FIG. 3A). FIG. 3B illustrates JP-col in LB agar, alongside solidified mixtures containing varying concentrations (0, 16, and 32 pM) of JP-col. Agar plates were prepared containing Janus Nanoparticles and antibiotics for testing their effect on bacterial growth.

The JP/Agar was prepared as follows: LB agar was prepared and sterilized according to standard protocols. A stock solution of JP-col was prepared. The JP Final Concentration was chose based upon the desired final concentration of JP in the agar (e.g., 32 pM). A JP volume that minimally affects the agar concentration (<20%) was preferred to avoid hindering bacterial growth. In this example, a JP stock solution was prepared that slightly exceeding the volume needed (e.g., prepare 220 μL JP-col for a desired 200 μL). If the JP is solubilized in ethanol, the ethanol was replaced. with nuclease-free water using centrifugation or another suitable method, before mixing with agar.

For better dispersion, the JP-col solution was sonicated in an ultrasonic bath (around 70° C.), avoiding excessive heating during sonication.

Combine JP with Agar: In a 5 mL tube, the calculated volume of JP solution (e.g., 200 μL JP-col) was added. While the tube was in the warm ultrasonic bath, the freshly prepared, warm, and liquid LB agar (e.g., 1300 μL) was added to the JP solution. The agar mixture was maintained at a warm temperature to prevent solidification. The LB agar/JP-col mixture was pipetted into a 96-well plate.

Antibiotics were incorporated into the dispensed LB agar/JP-col mixture, prior to cooling. Antibiotic dilutions were prepared beforehand. To assess the combined effect of JP and antibiotics, various antibiotic concentrations were added to designated wells. The antibiotic was gently mixed into the agar in each well to ensure even distribution and avoid bubbles. Control wells without JP or containing other control particles were also included. The agar mixture in the plate was then allowed to cool and solidify completely.

Wells were then inoculated with Bacteria. A 1:100 dilution of an overnight bacterial culture was prepared and 1 μL of the diluted bacterial suspension was added to each well. The plate was incubated at an appropriate temperature (30-37° C.) for bacteria growth, typically overnight.

The plates were observed and compared for bacterial growth across wells the following day.

### Example 3: Janus NP-Encapsulated Agar Gels Enhance Antibiotic Efficacy

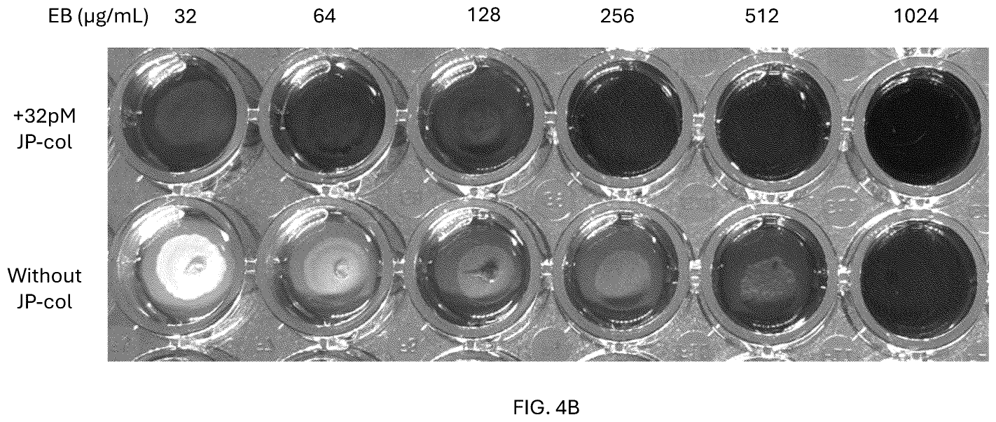

Our study demonstrated that agar gels encapsulating the hydrophobic/colistin Janus NPs (JP-col) significantly increase the susceptibility of both Escherichia coli and Acinetobacter baumannii to antibiotics. This synergistic effect was observed with ethidium bromide (EB) for E. coli (FIG. 4B) and cloxacillin (CLX) for A. baumannii (FIG. 4A). Our experiments measured the minimum concentration of antibiotics needed to inhibit bacterial growth (minimum inhibitory concentration, MIC). Notably, the incorporation of Janus NPs (JP-col) in agar gels significantly reduced the MIC of both antibiotics tested. In cultures of E. coli grown on agar, upon addition of the antibiotic ethidium bromide (EB), the MIC was extremely high, exceeding 1,024 μg/mL. However, on agar gel encapsulating 32 pM JP-col, the MIC dropped to 256 μg/mL, representing an 8-fold reduction (FIG. 4B). Similarly, for Acinetobacter baumannii grown on regular agar without Janus NPs, upon addition of the antibiotic cloxacillin (CLX), the initial MIC was 512 μg/mL. But on agar encapsulating 32 pM Janus NPs, the MIC decreased to a range of 128-256 μg/mL (FIG. 4A). Importantly, 32 pM JP-col alone didn't inhibit bacterial growth, as evidenced by observable growth at this concentration without antibiotics (FIGS. 4A and 4B). As shown in FIG. 4A, A. baumannii cells grown on JP-embedded agar show inhibited growth at lower concentrations of the antibiotic cloxacillin (CLX) compared to cells grown on regular agar. Without JP-col (bottom row) or with a lower JP-col concentration (middle row), some growth was observed at 128 μg/mL CLX. However, complete inhibition occurred at 256 μg/mL. In contrast, when 32 pM JP-col was encapsulated in the agar gel (top row), the MIC fell within a range of 128-256 μg/mL. Similarly, FIG. 4B for E. coli with ethidium bromide (EB) demonstrates an 8-fold reduction in MIC with 32 pM JP-col (top row) compared to no JP-col (bottom row). These results confirm that the Janus NP-embedded agar gels enhance the efficacy of antibiotics.

### Example 4: Effect of JNPs in Solution on Antibiotic Susceptibility in Bacteria

To assess the JNP-mediated potentiation of antibiotic susceptibility, we selected erythromycin (EM), a macrolide antibiotic and known efflux substrate, as a test compound. We selected four strains of A. baumannii-ATCC 19606 reference strain, BAA-1794 multidrug-resistant reference strain, and BAA-747 susceptible reference strain, and A42-2 drug-resistant clinical isolate. Similar tests were also conducted with antimicrobials CIP, ciprofloxacin; AMK, amikacin; MER, meropenem; MOX, moxifloxacin; CLX, cloxacillin; EM, erythromycin; RIF, rifampicin; TET, tetracycline; and PB, polymyxin B. (Table 1)

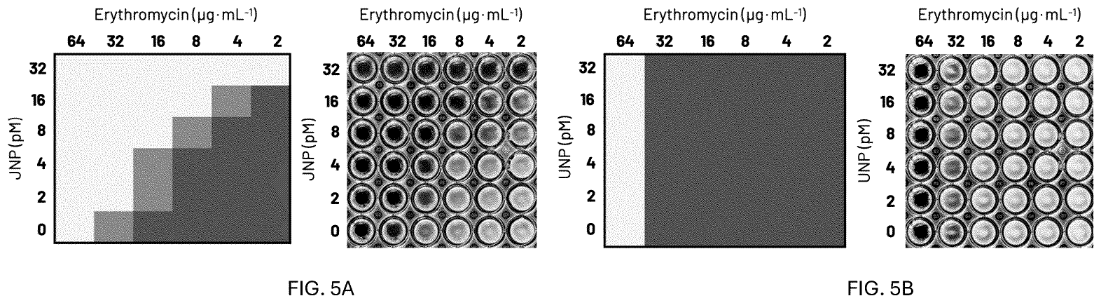

We first confirmed that JNPs alone had no antimicrobial activity against A. baumannii, as they did not inhibit cell growth at concentrations up to at least 64 pM (FIG. 5A). Minimum inhibitory concentration (MIC) assays performed on solid agar showed consistent results across replicates: the MIC of EM was 4 μg/mL for the susceptible reference strain BAA-747, 16 μg/mL for ATCC 19606, and 32 μg/mL for the drug-resistant clinical isolate A42-2 (Table 1).

TABLE 1
Antimicrobial susceptibility of clinical and reference A. baumannii strains

| | | Minimum inhibitory concentration (MIC) (mg mL⁻¹) | | | | | | | | |
|---|---|---|---|---|---|---|---|---|---|---|
| # | Strain | CIP | AMK | MER | MOX | CLX | EM | RIF | TET | PB |
| 1 | 19606 | 0.5 | 8-16 | 1-2 | 0.25 | 1024 | 16 | 1-2 | 2-4 | 1 |
| 2 | 1794 | 64 | 64-128 | 64 | 4 | 1024 | 8 | 2-4 | >128 | 1 |
| 3 | 747 | ≤0.125 | 2 | 0.25-0.5 | ≤0.0313 | >1024 | 4 | 4 | 4 | 1 |
| 4 | A42-2 | 128-256 | 32-128 | 8 | 32-64 | >1024 | 32 | 2-4 | >128 | 1 |

To assess potential synergy between JNP and erythromycin (EM), we conducted a checkerboard assay in liquid broth using A. baumannii ATCC 19606. JNP was serially diluted in two-fold steps up to a maximum concentration of 32 pM. The resulting checkerboard matrix (FIG. 5A), with EM concentrations along the x-axis and JNP concentrations along the y-axis, revealed a clear pattern of growth inhibition, indicating that JNP enhances the susceptibility of A. baumannii ATCC 19606 to EM. JNP significantly reduced the effective MIC of erythromycin from 64 μg·mL⁻¹ to 8 μg·mL⁻¹ at 16 pM, and to <2 μg/mL at 32 pM JNP.

To determine whether this potentiating effect is specific to the Janus structure of the nanoparticles, we compared JNP to uniformly coated colistin nanoparticles (UNP) under identical conditions. Unlike JNP, UNP did not alter the antibiotic susceptibility of ATCC 19606 (FIG. 5B), indicating that the unique partial colistin coating of JNP is critical for its synergistic activity.

The fractional inhibitory concentration index (FICI) is a standard metric used to assess the interaction between two antimicrobial agents. It is calculated as the sum of the MICs of each agent in combination, normalized to their respective MICs when used alone: FICI=(MIC of antibiotic in combination/MIC of antibiotic alone)+(MIC of JNP in combination/MIC of JNP alone). According to widely accepted criteria, a FICI≤0.5 indicates synergy, values between 0.5 and 4.0 indicate no interaction (or indifference), and values >4.0 suggest antagonism.

Based on the data shown in FIG. 5A, the MIC of EM alone against A. baumannii ATCC 19606 was 64 μg/mL. In combination with JNPs, the MIC of EM was reduced to 8 μg/mL in the presence of 16 pM JNP. This corresponds to a FICI of 0.5 when assuming a JNP MIC of 64 pM, and a more conservative estimate of 0.25 if the MIC of JNP is considered to be >64 pM. Both values fall within the synergistic range. Similarly, at 8 pM JNP, the FICI ranged from 0.3125 to 0.375, further supporting synergy. These results were consistent across multiple experimental replicates.

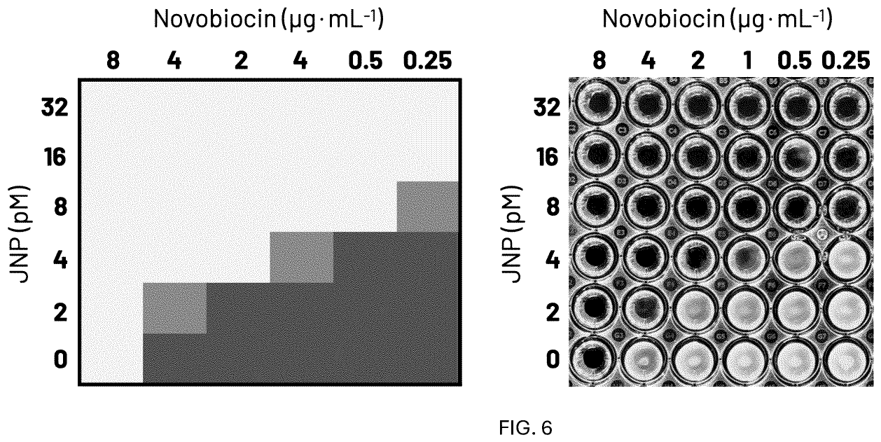

To further evaluate the specificity of JNP and its ability to potentiate other antibiotics, we tested the aminocoumarin antibiotic novobiocin (NOV). In liquid broth assays, the MIC of NOV against A. baumannii ATCC 19606 ranged from 4 to 16 μg/mL, depending on the experimental replicate. JNP significantly reduced these MIC values at concentrations as low as 2-4 pM, as shown across multiple experiments (FIG. 6). In the presence of 16 pM JNP, the MIC of NOV decreased from 8 to <0.25 μg/mL. The calculated FICI was 0.3125 at 4 pM JNP and 0.1875 at 8 pM, both indicating strong synergistic interactions. In contrast, uniformly coated colistin nanoparticles (UNP) had no effect on NOV susceptibility, reinforcing the structural specificity of JNP.

Checkerboard assays with erythromycin (EM) and novobiocin (NOV), along with additional tests using rifampicin (RIF), kanamycin (KAN), and ethidium bromide (EtBr), consistently showed that JNP reduced the MIC values of all tested compounds against A. baumannii ATCC 19606. These results indicate that JNP broadly enhances the antibiotic susceptibility of A. baumannii. In contrast, uniformly coated colistin nanoparticles (UNP) did not alter the susceptibility to any of the tested agents, reinforcing that the synergistic effect is specific to the unique Janus structure of JNP.

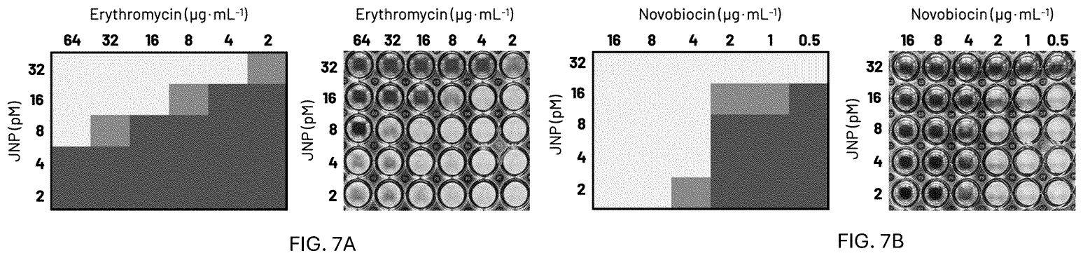

We also assessed the effect of JNP on the drug susceptibility of the clinical isolate A42-2, which exhibited substantially higher resistance to all tested antibiotics compared to A. baumannii ATCC 19606 (Table 1). Results are presented in FIG. 7A and FIG. 7B. To ensure comparability and verify JNP activity, checkerboard assays for A42-2 were performed in parallel with those for ATCC 19606. Erythromycin (EM) and novobiocin (NOV) were selected for these assays, as they had the most complete replicate data for ATCC 19606. Given the increased drug resistance of A42-2, JNP was tested at concentrations up to 64 pM. In both EM and NOV assays, JNP significantly reduced the MICs for A42-2, confirming its synergistic effect even in this highly drug-resistant, clinically relevant strain (FIG. 7A and FIG. 7B). The FICI for EM was 0.375 at 32 pM JNP (assuming a MIC of 128 μg/mL), while for NOV the FICI was 0.5625 at both 8 and 64 pM JNP. Although the NOV FICI slightly exceeds the synergy threshold (≤0.5), it still suggests a potentiating interaction. In contrast, uniformly coated colistin nanoparticles (UNP) had no effect on A42-2 susceptibility to either EM or NOV, further supporting the structural specificity of JNP.

Given the significant potentiating effects of JNP, but not UNP, on antibiotic susceptibility in A. baumannii, including both a reference strain and a multidrug-resistant clinical isolate, we next investigated whether similar synergistic effects could be observed in Escherichia coli. For this purpose, we selected E. coli K-12 MG1655, a model Gram-negative strain with well-characterized resistance mechanisms and susceptibility profiles. MG1655 is among the most extensively studied E. coli strains and was the first in the species to have its complete genome sequenced. Multidrug-resistant E. coli is classified by the World Health Organization (WHO) as a high-priority pathogen. It falls within the “critical” priority group of Enterobacterales (which includes E. coli and Klebsiella pneumoniae), with third-generation cephalosporin-resistant E. coli ranked just above A. baumannii on the WHO's list. Similarly, the U.S. Centers for Disease Control and Prevention (CDC) classifies carbapenem-resistant Enterobacterales (CRE), including carbapenem-resistant E. coli (CREC), as an “urgent threat”.

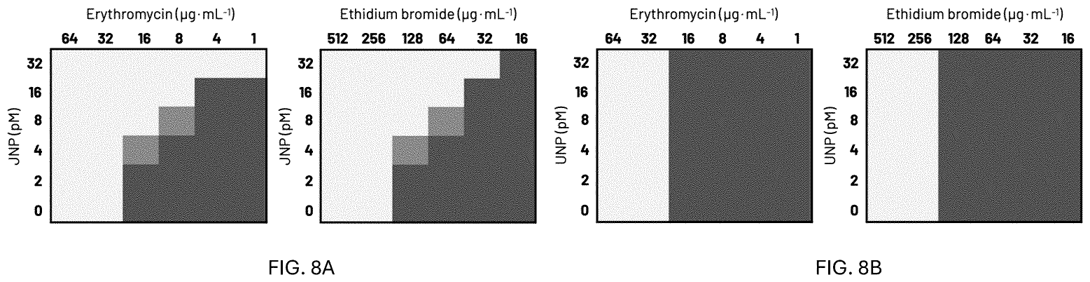

To assess whether JNP could enhance antibiotic susceptibility in E. coli, we selected crythromycin (EM), for which we had the most comprehensive data in A. baumannii. Checkerboard assays were performed using E. coli MG1655 in combination with JNP or UNP. We also tested ethidium bromide (EtBr) and novobiocin (NOV). Representative results for EM and EtBr are shown in FIG. 8A and FIG. 8B. For EM, the FICI values were 0.375 at 16 pM JNP and ≤0.313 at 32 pM, indicating synergy. For EtBr, FICI values were 0.375 at both 16 pM and 32 pM JNP. These results demonstrate that JNP enhances the susceptibility of E. coli MG1655 to multiple compounds, consistent with its effects in A. baumannii.

### Example 5: Effect of Embedding JNPs in Agar Gel on Antibiotic Susceptibility in Bacteria for Slow Acting Antibiotics

By positioning bacterial cells on the surface of JNP-containing agar, we promoted consistent, localized interaction, especially important for motile organisms like E. coli, which can otherwise avoid contact with nanoparticles in suspension. This solid-phase approach also offered an additional advantage: it potentially enhances the efficacy of JNPs when combined with slow-acting antibiotics, such as β-lactams, which represent a major class of clinically used drugs. In liquid culture, even high concentrations of β-lactams like cloxacillin (CLX) permit bacterial growth for several hours before bactericidal effects occur. Stable, sustained exposure to JNPs in agar may accelerate antibiotic activity, reducing this delay.

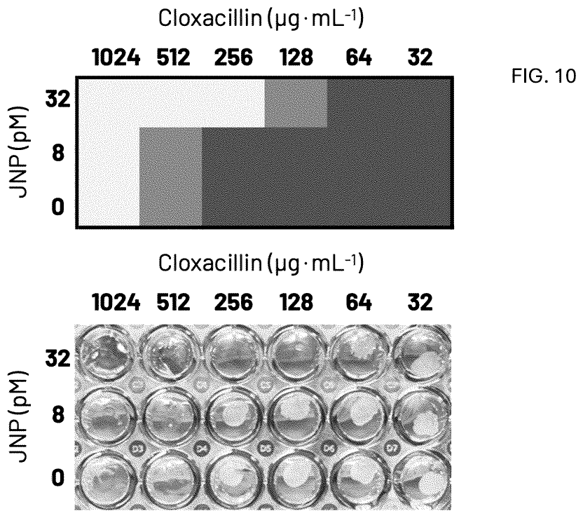

To test this hypothesis, we examined the effect of JNPs on β-lactam susceptibility by combining 0, 8, or 32 pM JNP with CLX against A. baumannii ATCC19606. As noted earlier, β-lactam resistance in A. baumannii poses a major global health threat. Without JNP, the MIC of CLX was 1024 μg·mL⁻¹. The addition of 8 pM JNP had no effect, but at 32 pM, the MIC dropped to 256 μg·mL⁻¹, a fourfold reduction, indicating a significant increase in susceptibility (FIG. 10).

Next, we investigated whether the agar-based method could enhance the effectiveness of JNPs in sensitizing E. coli to antibiotics. We selected ethidium bromide (EtBr) for this assay, same as done in liquid media. Using the solid agar approach, we repeated the assays at least three times and obtained reproducible results. A representative outcome is shown in FIG. 11. The consistent and pronounced growth inhibition observed on JNP-supplemented agar highlights the efficacy of this delivery method. These results suggest that even localized, surface-level contact with JNPs embedded in the agar matrix is sufficient to compromise the integrity of E. coli and A. baumannii cell envelopes. Remarkably, this effect parallels that of JNPs in suspension, where nanoparticles can interact with bacterial cells from all directions.

### Materials and Methods Used in the Examples

Cells and reagents. E. coli (MG1655), S. aureus (Newman), B. subtilis (SB168) and V. cholerae (unpublished strain) were generously provided by the Gerdt lab at Indiana University-Bloomington. Luria-Bertani (LB) agar and Luria broth base were purchased from Invitrogen (Waltham, MA, USA). Amine-functionalized silica nanoparticles (100 nm) were procured from Nanocomposix (San Diego, CA, USA). Gold (99.99% purity) and chromium (99.99% purity) were purchased from Kurt J. Lesker, Co. (Jefferson Hills, PA, USA). Octadecanethiol, propidium iodide, octadecyltrimethoxysilane, colistin sulfate, G1 poly(amidoamine) dendrimer (20 wt % in methanol), and HEPES were purchased from Sigma-Aldrich (St. Louis, MO, USA). Ultrapure water (18 M (2·cm) was used for all experiments.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed. Unless states otherwise of clearly implied otherwise the term ‘about’ means plus or minus 10 percent, for example about 1.0 encompasses the ranges of values from 0.9 to 1.1.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications and references is hereby incorporated by reference, particularly for the teaching referenced herein.

I claim:

1. An antimicrobial material, comprising:
a Janus nanoparticle;
a first antibiotic; and
a cross-linked polymer,
wherein the Janus nanoparticle comprises a nanoparticle having a plurality of hydrophobic moieties attached to a first hemisphere of the nanoparticle and a plurality of charged moieties attached to the opposite hemisphere of the nanoparticle, and wherein the Janus nanoparticle is embedded within the cross-linked polymer.

2. The antimicrobial material according to claim 1, wherein hydrophobic moiety comprises at least one hydrophobic alkyl chain.

3. The antimicrobial material according to claim 2, wherein the at least one hydrophobic alky chain is selected from the group consisting of: linear chains, branched chains, or polymers.

4. The antimicrobial material according to claim 1, wherein the charged moiety is a second antibiotic.

5. The antimicrobial material according to claim 4, wherein the second antibiotic is selected from the group consisting of: polymyxin antibiotics, antimicrobial peptides, and steroid antibiotic, wherein the antibiotic exhibits a positive charge.

6. The antimicrobial material according to claim 5, wherein the antibiotic is colistin.

7. The antimicrobial material according to claim 1 wherein the cross-linked polymer is biocompatible.

8. The antimicrobial material according to claim 1, wherein the first antibiotic compound is selected from the group consisting of ciprofloxacin (CIP), amikacin (AMK), meropenem (MER), moxifloxacin (MOX), cloxacillin (CLX), erythromycin (EM), rifampicin (RIF), tetracycline (TET), ethidium bromide (EB), novobiocin (NOV) and polymyxin B (PB).

9. The antimicrobial material according to claim 1 wherein the concentration of antibiotic added is 0.5 to 0.01 the minimum inhibitory concentration (MIC) (mg/mL) of the concentration of antibiotic needed to reach the same MIC alone.

10. The antimicrobial material according to claim 1, wherein the antimicrobial material kill Gram-negative bacteria.

11. The antimicrobial material according to claim 1, wherein the antimicrobial material kill Gram-positive bacteria.

12. A wound dressing incorporating the antimicrobial material of claim 1.

13. A method of administering an antimicrobial to a subject in need thereof, the method comprising administering to said subject an antimicrobial material comprising
a Janus nanoparticle;
a first antibiotic; and
a cross-linked polymer,
wherein the Janus nanoparticle comprises a nanoparticle having a plurality of hydrophobic moieties attached to a first hemisphere of the nanoparticle and a plurality of charged moieties attached to the opposite hemisphere of the nanoparticle, and wherein the Janus nanoparticle is embedded within the cross-linked polymer.

14. The method of claim 13, wherein the antimicrobial material is applied topically to the subject.

15. The method of claim 14, wherein the antimicrobial material is applied topically to a surgical wound.

16. The method of claim 14, wherein the antimicrobial material is applied topically to a bacterial infection present on the skin of a subject.

17. The method according to claim 13, wherein the therapeutically effective dose of Janus nanoparticles contained in the antimicrobial material is within the picomolar to micron-molar range.

18. The method according to claim 13, wherein the bacterial infection is caused by at least one bacterium selected from the group consisting of: Gram-negative and Gram-positive bacteria.

19. The methods according to claim 13, wherein the patient is a human or an animal.
